(19) United States
(12) Patent Application Publication
(10) Pub. No.: US 2023/0181166 A1
(43) Pub. Date: Jun. 15, 2023

(54) METHOD AND SYSTEM FOR CAPTURING AND SUMMARIZING CHANGES IN ULTRASOUND SCANNING SETTINGS

(71) Applicant: GE Precision Healthcare LLC, Wauwatosa, WI (US)

(72) Inventors: Jenny Pagano, St. Georgen im Attergau (AT); Christian Perrey, Mondsee (AT)

(21) Appl. No.: 17/551,785

(22) Filed: Dec. 15, 2021

(51) Int. Cl. A61B 8/08; A61B 8/00; G16H 30/40

(52) U.S. Cl. CPC A61B 8/5292; A61B 8/467; A61B 8/5253; A61B 8/5223; G16H 30/40; H04N 5/262

(57) ABSTRACT

Systems and methods for detecting changes to ultrasound scanning settings during an ultrasound examination and generating a record of the settings changes are provided. The method includes acquiring, by an ultrasound system, ultrasound image data according to ultrasound scanning settings during an ultrasound examination. The method includes monitoring, by at least one processor, the ultrasound scanning settings during the ultrasound examination and detecting a changed ultrasound scanning setting from the ultrasound scanning setting during the ultrasound examination. The method includes recording, by the at least one processor, the changed ultrasound scanning setting in a log file associated with the ultrasound examination. The method includes recording, by the at least one processor, a video clip of the ultrasound image data a predetermined period of time before and after the changed ultrasound scanning setting is detected. The video clip is associated with the ultrasound examination.

### FIELD

Certain embodiments relate to ultrasound imaging. More specifically, certain embodiments relate to a method and system for detecting changes to ultrasound scanning settings during an ultrasound examination and generating a record of the settings changes. The record may comprise a log file of the settings changes and/or a video clip of acquired ultrasound images spanning a predetermined period of time before and after the settings change.

### BACKGROUND

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

Technologies for automatically optimizing certain imaging settings during an ultrasound examination have been emerging for ultrasound machines. Examples of imaging settings that may be automatically optimized during an ultrasound examination include gain, depth, frequency, gray map settings, compound resolution imaging (CRI) settings, region of interest (ROI) box placement, acoustic output (AO), pulsed Doppler (PD) gate placement, and the like. The automatic optimization of the imaging settings typically occurs without the ultrasound operator being aware of the specific settings changes being made. Accordingly, ultrasound operators may be unaware of how specific settings changes may affect the visualization of the ultrasound images and/or the level of impact of specific settings changes.

Ultrasound machines may be set-up or updated by application specialists to include presets for ultrasound operators. However, the application specialists may be unaware of manual imaging settings changes performed by various ultrasound operators and/or automatic imaging settings changes performed by the ultrasound machine during ultrasound examinations.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for detecting changes to ultrasound scanning settings during an ultrasound examination and generating a record of the settings changes, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to detect changes to ultrasound scanning settings during an ultrasound examination and generate a record of the settings changes, in accordance with various embodiments.

FIG. 2 illustrates an exemplary log file that may be generated by an ultrasound system to track changes to ultrasound scanning settings during an ultrasound examination, in accordance with various embodiments.

FIG. 3 illustrates a screenshot of a display system presenting exemplary ultrasound image data acquired before and after a change to an ultrasound scanning frequency setting during an ultrasound examination, in accordance with various embodiments.

FIG. 4 illustrates a screenshot of a display system presenting exemplary ultrasound image data acquired before and after a change to an ultrasound scanning speckle reduction imaging (SRI) setting during an ultrasound examination, in accordance with various embodiments.

FIG. 5 illustrates a screenshot of a display system presenting exemplary ultrasound image data acquired before and after a change to an ultrasound scanning pulse repetition frequency (PRF) setting during an ultrasound examination, in accordance with various embodiments.

FIG. 6 is a flow chart illustrating exemplary steps for detecting changes to ultrasound scanning settings during an ultrasound examination and generating a record of the settings changes, in accordance with various embodiments.

### DETAILED DESCRIPTION

Certain embodiments may be found in a method and system for detecting changes to ultrasound scanning settings during an ultrasound examination and generating a record of the settings changes. Various embodiments have the technical effect of providing a record associated with an ultrasound examination documenting ultrasound scanning settings changes during the ultrasound examination. The record may comprise a log file listing the ultrasound scanning settings changes. The record may comprise video clips of acquired ultrasound image data spanning a predetermined time period before and after the ultrasound scanning settings changes. Aspects of the present disclosure have the technical effect of assigning an impact level to each ultrasound scanning settings change based on contextual information, and providing the impact level in the record. Certain embodiments have the technical effect of providing a machine readable log file that may be loaded into an ultrasound system to create and/or modify ultrasound imaging presets. Various embodiments have the technical effect of stitching together video clips to show all ultrasound scanning settings changes during an ultrasound examination. Aspects of the present disclosure have the technical effect of annotating a video clip of ultrasound scanning settings changes during an ultrasound examination with descriptions of the changes made and/or the impact level of the changes.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, Contrast Enhanced Ultrasound (CEUS), and/or sub-modes of B-mode and/or CF such as Harmonic Imaging, Shear Wave Elasticity Imaging (SWEI), Strain Elastography, TVI, PDI, B-flow, MVI, UGAP, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

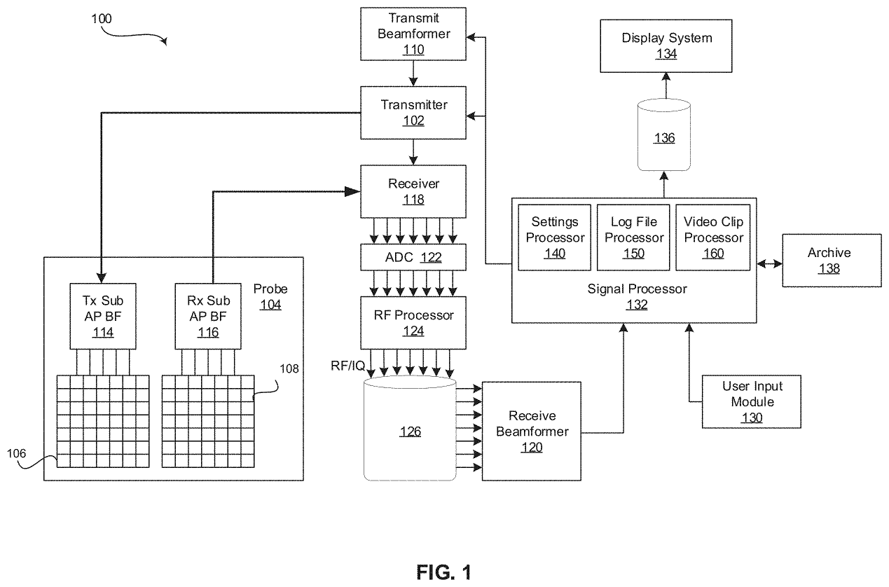

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to detect changes to ultrasound scanning settings during an ultrasound examination and generate a record of the settings changes, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiments, the ultrasound probe 104 may be operable to acquire ultrasound image data covering at least a substantial portion of an anatomy, such as the heart, a blood vessel, a fetus, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, input or change ultrasound scanning settings, select presets, protocols, and/or templates, select records of ultrasound scanning settings changes for retrieval and display, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, graphic processing units, microprocessors, microcontrollers, and/or the like. The signal processor 132 may be an integrated component, or may be distributed across various locations, for example. In an exemplary embodiment, the signal processor 132 may comprise a settings processor 140, a log file processor 150, and a video clip processor 160 and may be capable of receiving input information from a user input device 130 and/or archive 138, generating an output displayable by a display system 134, and manipulating the output in response to input information from a user input device 130, among other things. The signal processor 132, settings processor 140, log file processor 150, and video clip processor 160 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a settings processor 140 that comprises suitable logic, circuitry, interfaces and/or code that may be operable to monitor ultrasound scanning settings during an ultrasound examination to detect and changes to the ultrasound scanning settings. The settings processor 140 may be configured to detect initial ultrasound scanning settings, such as settings associated with a selected preset or otherwise provided by an ultrasound operator via the user input device 130. The settings processor 140 may be configured to detect ultrasound scanning settings changes provided by the ultrasound system 100 when operating in an automatic optimization mode during an ultrasound examination. The settings processor 140 may be configured to detect ultrasound scanning settings changes provided manually by an ultrasound operator via the user input device during an ultrasound examination. The settings processor 140 may be configured to provide the detected initial ultrasound scanning settings and the detected settings changes to the log file processor 150. The settings processor 140 may be configured to notify the video clip processor 160 of ultrasound scanning settings changes. In various embodiments, the settings processor 140 may selectively monitor ultrasound scanning settings based on whether the ultrasound scanning settings changes detection and recording feature is in an on state or an off state. For example, an ultrasound operator may turn on the ultrasound scanning settings changes detection and recording feature manually, such as to detect and record changes during short sequences of the ultrasound examination for teaching purposes. As another example, the ultrasound scanning settings changes detection and recording feature may be automatically placed in an on state based on pre-defined ultrasound operator selections, such as an ultrasound operator switching to a heart imaging preset.

The signal processor 132 may include a log file processor 150 that comprises suitable logic, circuitry, interfaces and/or code that may be operable to generate a log file associated with an ultrasound examination and record initial ultrasound scanning settings and settings changes made during the ultrasound examination. For example, the log file processor 150 may receive the initial ultrasound scanning settings selected at the onset of an ultrasound examination from the settings processor 140. The log file processor 150 may be configured to generate a log file and associate the log file with the ultrasound examination. The log file processor 150 may be configured to record ultrasound examination information and the initial ultrasound scanning settings in the log file. The ultrasound examination information may include ultrasound machine information, ultrasound probe information, the ultrasound operator, the ultrasound examination date and time, and the like. The initial ultrasound scanning settings may include a selected preset, a frequency setting, a gain setting, a depth setting, and any suitable initial settings selected by an ultrasound operator and/or included in a selected preset. The log file processor 150 may receive ultrasound scanning settings changes throughout the ultrasound examination from the settings processor 140. The log file processor 150 may be configured to record each of the ultrasound scanning settings changes in the log file. The record of each of the ultrasound scanning settings changes may include a time the settings change was made, an identification of the changed setting and the changed setting value, an assigned impact level of the settings change to an accuracy of a diagnosis, and a list of the settings after the change was made. In various embodiments, the log file may be machine readable, such that the log file may be applied to an ultrasound system 100 to create new presets and/or modify existing presets. In an exemplary embodiment, the log file processor 150 may receive inputs from an ultrasound operator via the user input device 130 to mark the log file, such that the machine readable log file can distinguish between different tasks, and the settings applied for each of the marked tasks, performed during an ultrasound examination. The log file may be stored in archive 138 and/or any suitable data storage medium.

The log file processor 150 may comprise suitable logic, circuitry, interfaces and/or code that may be operable to determine and assign the impact level of the settings change to an accuracy of a diagnosis based on the particular setting being changed and contextual information related to the ultrasound examination. For example, the impact level to an accuracy of a diagnosis may be categories (e.g., low, medium, high), number values (e.g., 1-5), letter values/grades (e.g., A-F), or any suitable level characterization. As an example, a speckle reduction imaging (SRI) setting value change may generally be assigned a lesser impact (e.g., low, 1, A, or the like) because a change in SRI is typically an aesthetic preference that has less effect on an ability of an ultrasound operator or other medical professional to provide an accurate diagnosis based on the ultrasound image data being presented at the display system 134. However, if the contextual information indicates that a typical measurement is being performed, the SRI setting value may have a greater effect on an ability to perform the measurement and thereby provide an accurate diagnosis, causing the log file processor 150 to assign a higher impact level (e.g., medium, 3, C, or the like). As another example, frequency settings, pulse repetition frequency (PRF) settings, flow settings (e.g., HD flow, slow flow, etc.), and the like may have higher impact levels (e.g., medium or high; 3, 4, or 5; C, D, or F; or the like) because these settings have a larger effect on the ultrasound image data being presented at the display system 134 and an ability of an ultrasound operator or other medical professional to visualize needed information to provide an accurate diagnosis. Examples of contextual information that may be applied by the log file processor 150 with the identity of the setting being changed to determine the impact level may include, an imaging mode, a selected measurement, and/or any suitable ultrasound examination contextual information. The log file processor 150 may record the assigned impact level of the particular ultrasound scanning settings change in the log file. In various embodiments, the log file processor 150 may be configured to provide the assigned impact level to the video clip processor 160 for annotating the video clip. In certain embodiments, the log file processor 150 may be configured to selectively record ultrasound scanning settings changes based on a pre-defined filter criteria. For example, the log file processor 150 may be configured to only record ultrasound scanning settings changes assigned a high impact level, or any suitable pre-defined filter criteria.

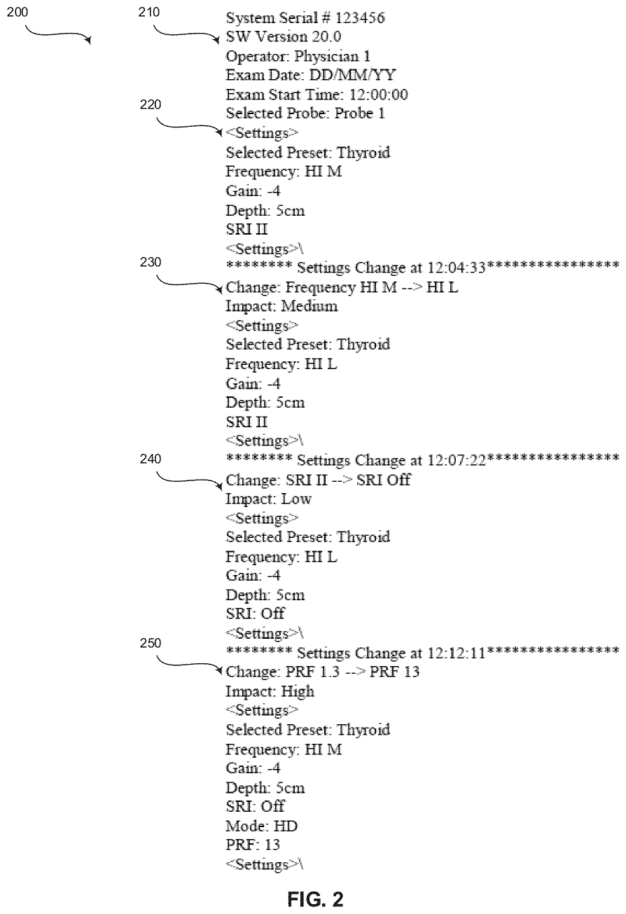

FIG. 2 illustrates an exemplary log file 200 that may be generated by an ultrasound system 100 to track changes to ultrasound scanning settings during an ultrasound examination, in accordance with various embodiments. Referring to FIG. 2, the log file 200 generated by the log file processor 150 of the ultrasound system 100 may comprise ultrasound examination information 210, initial ultrasound scanning settings 220, and ultrasound scanning settings changes 230-250 received from the settings processor 140 before and during an ultrasound examination. The ultrasound examination information 210 may include ultrasound system 100 identification information, a software version executed by the ultrasound system 100, the ultrasound operator, the ultrasound examination date and time, ultrasound probe 104 information, and the like. The initial ultrasound scanning settings 220 may include a selected preset, a frequency setting, a gain setting, a depth setting, a speckle reduction imaging (SRI) setting, and any suitable initial settings selected by an ultrasound operator and/or included in a selected preset. The ultrasound scanning settings changes 230-250 may include a time the settings change was made, an identification of the changed setting and the changed setting value, an assigned impact level of the settings change to an accuracy of a diagnosis, and a list of the settings after the change was made. For example, FIG. 2 illustrates a first settings change 230 from a medium frequency level (e.g., HI M) to a low frequency level (e.g., HI L). The log file processor 150 assigned a medium impact level to an accuracy of a diagnosis based on the first settings change 230 from the medium frequency level to the low frequency level in view of the ultrasound examination contextual information (e.g., imaging mode, selected measurement, etc.). The second settings change 240 corresponds with a change detected by the settings processor 140 from a speckle reduction imaging (SRI) level II to SRI being turned off. The log file processor 150 assigned a low impact level to an accuracy of a diagnosis based on the settings change 240 from the SRI II level to the SRI Off level in view of the ultrasound examination contextual information (e.g., imaging mode, selected measurement, etc.). The third settings change 250 corresponds with a change detected by the settings processor 140 from a pulse repetition frequency (PRF) of 1.3 to a PRF of 13. The log file processor 150 assigned a high impact level to an accuracy of a diagnosis based on the settings change 250 from the PRF value of 1.3 to the PRF value of 13 in view of the ultrasound examination contextual information (e.g., imaging mode, selected measurement, etc.). Although three (3) settings changes 230-250 are illustrated in the log file 200 of FIG. 2, more or less ultrasound scanning settings changes 230-250 are contemplated depending on the number of ultrasound settings changes 230-250 detected by the settings processor 140 during an ultrasound examination.. In various embodiments, the log file 200 may be machine readable, such that settings in the log file 200 may be applied to an ultrasound system 100 to create new presets and/or modify existing presets. The log file 200 generated by the log file processor 150 may be stored in archive 138 and/or any suitable data storage medium.

Referring again to FIG. 1, the signal processor 132 may include a video clip processor 160 that comprises suitable logic, circuitry, interfaces and/or code that may be operable to record a video clip spanning a predetermined period of time before and after each ultrasound scanning settings change 230-250 detected by the settings processor 140. For example, the video clip processor 160 may receive an indication of a settings change from the settings processor 140 and in response, may record a video clip that begins 1-3 seconds (or any suitable defined period of time) before the settings change and ends 1-3 seconds (or any suitable defined period of time) after the settings change. The video clip processor 160 may be configured to stitch together each video clip corresponding with each ultrasound scanning settings change to generate a continuous video clip illustrating all of the ultrasound scanning settings changes during an ultrasound examination. In various embodiments, the video clip processor 160 may comprise suitable logic, circuitry, interfaces and/or code that may be operable to annotate the video clip(s) with the ultrasound scanning settings change, the assigned impact level, and/or any suitable information. The resulting video clip and/or the individual video clips may be associated with the ultrasound examination. The resulting video clip and/or the individual video clips may be stored at archive 138 and/or any suitable data storage medium. The resulting video clip and/or the individual video clips may be selectively displayed in response to an ultrasound operator instruction via the user input device 130. The resulting video clip and/or the individual video clips may be presented at the display system 134 separately and/or along with a retrieved log file 200. In various embodiments, the video clip processor 160 may be configured to selectively record ultrasound scanning settings changes based on a pre-defined filter criteria. For example, the video clip processor 160 may be configured to only record ultrasound scanning settings changes in response to an indication from the log file processor 150 that the setting change was assigned a high impact level, or any suitable pre-defined filter criteria.

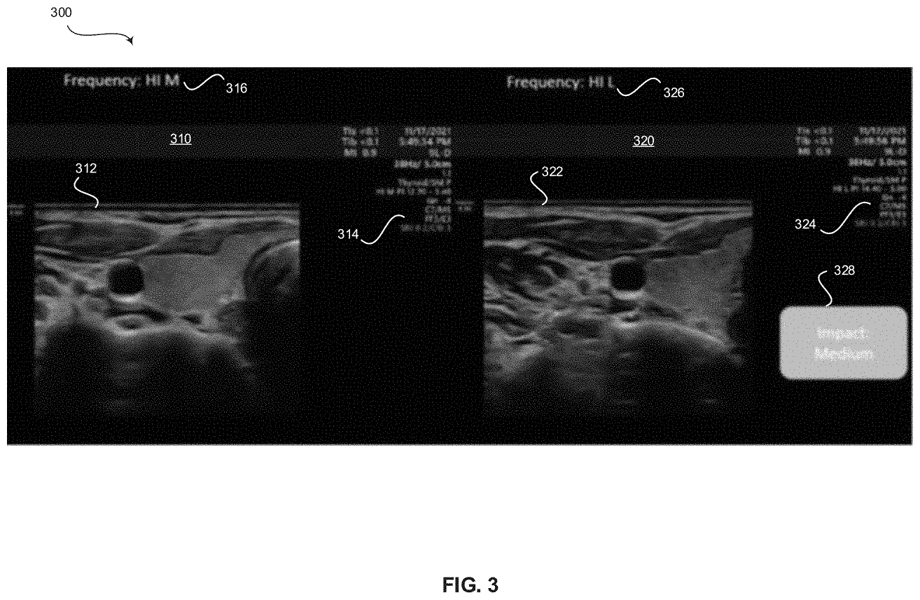

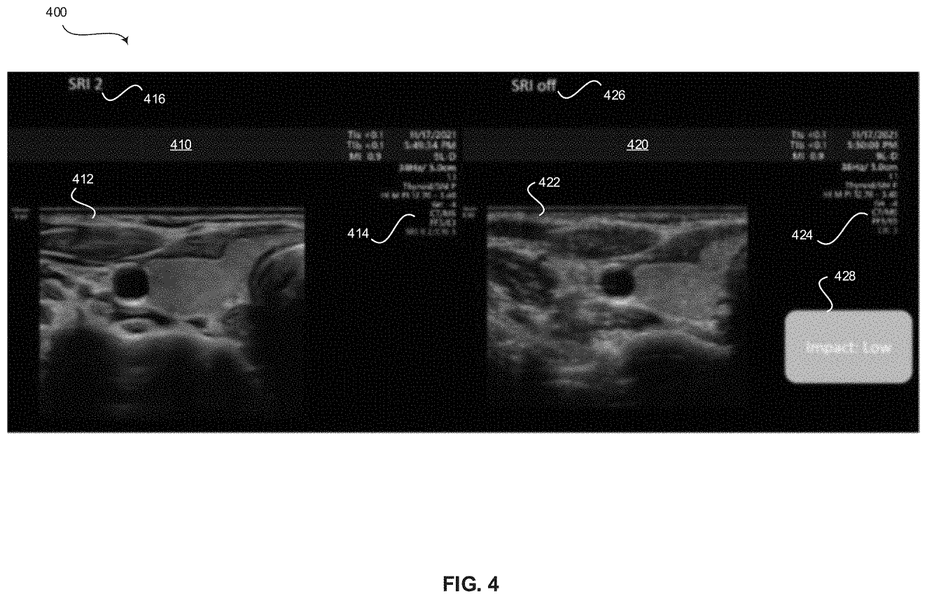

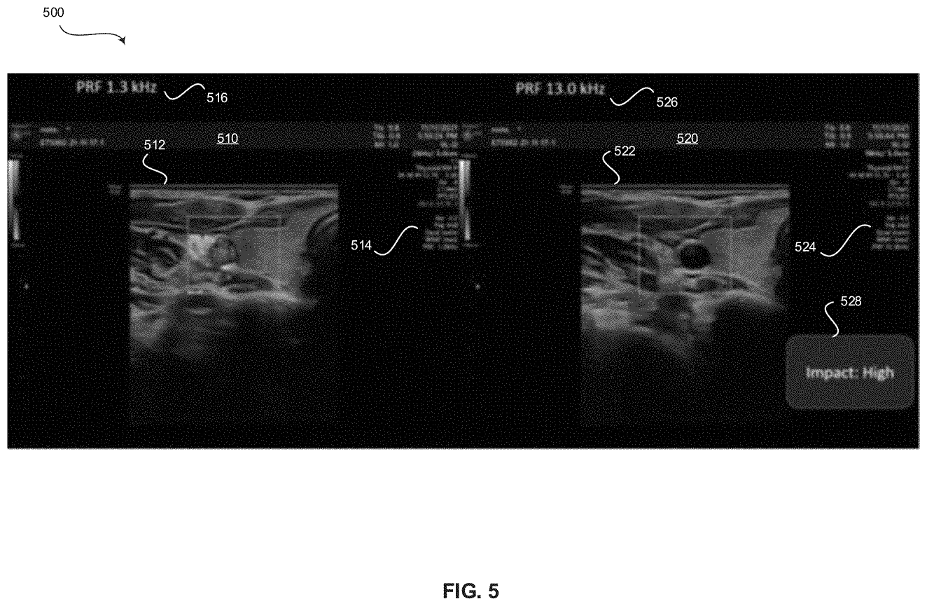

FIG. 3 illustrates a screenshot 300 of a display system 134 presenting exemplary ultrasound image data 312, 322 acquired before 310 and after 320 a change to an ultrasound scanning frequency setting 316, 326 during an ultrasound examination, in accordance with various embodiments. FIG. 4 illustrates a screenshot 400 of a display system 134 presenting exemplary ultrasound image data 412, 422 acquired before 410 and after 420 a change to an ultrasound scanning speckle reduction imaging (SRI) setting 416, 426 during an ultrasound examination, in accordance with various embodiments. FIG. 5 illustrates a screenshot 500 of a display system 134 presenting exemplary ultrasound image data 512, 522 acquired before 510 and after 520 a change to an ultrasound scanning pulse repetition frequency (PRF) setting 516, 526 during an ultrasound examination, in accordance with various embodiments. Referring to FIGS. 3-5, the screenshots 300, 400, 500 comprise a video clip of acquired ultrasound image data 312, 412, 512 before an ultrasound scanning settings change 310, 410, 510 and a video clip of acquired ultrasound image data 322, 422, 522 after an ultrasound scanning settings change 320, 420, 520. In various embodiments, a single video clip of acquired ultrasound image data 312, 322, 412, 422, 512, 522 is displayed. The screenshots 300, 400, 500 illustrating both before 310, 410, 510 and after 320, 420, 520 the ultrasound scanning settings changes is provided for ease of explanation and to show the appearance of the information presented at the display system 134 both before 310, 410, 510 and after 320, 420, 520 the setting change. In other words, the left (i.e., before) 310, 410, 510 side of each screenshot 300, 400, 500 is presented by the display system 134 until the settings change occurs, at which time, the right (i.e., after) 320, 420, 520 side of each screenshot 300, 400, 500 is presented by the display system 134. In an exemplary embodiment, the video clips 312, 322, 412, 422, 512, 522 may be stitched together by the video clip processor 150 to provide a single video clip of all settings changes made during an ultrasound examination.

The screenshots 300, 400, 500 illustrated in FIGS. 3-5 correspond with the settings changes 230-250 identified in the log file of FIG. 2. For example, FIG. 3 illustrates the video clip 312, 322 of the ultrasound scanning settings change 230 from a medium frequency level (i.e., HI M) to a low frequency level (i.e., HI L). FIG. 4 illustrates the video clip 412, 422 of the ultrasound scanning settings change 240 from a speckle reduction imaging (SRI) level II to an SRI level Off. FIG. 5 illustrates the video clip 512, 522 of the ultrasound scanning settings change 250 from a 1.3 kHz pulse repetition frequency (PRF) to a 13 kHz PRF. The screenshots 300, 400, 500 of FIGS. 3-5 are annotated with the ultrasound scanning setting 316, 326, 416, 426, 516, 526 that are changed in the video clip 312, 322, 412, 422, 512, 522. The screenshots 300, 400, 500 of FIGS. 3-5 are further annotated with the impact level 328, 428, 528 determined by the log file processor 150 and corresponding to the ultrasound scanning settings change in view of the ultrasound examination contextual information (e.g., imaging mode, selected measurement, etc.). The screenshots 300, 400, 500 of FIGS. 3-5 may further include ultrasound examination information and/or at least some of the ultrasound scanning settings 314, 324, 414, 424, 514, 524. The video clip 312, 322, 412, 422, 512, 522 of the acquired ultrasound image data a pre-determined amount of time (e.g., 1-3 seconds) before 310, 410, 510 and after 320, 420, 520 the ultrasound scanning settings changes 230-250 allow an ultrasound operator to visualize the effect of the settings changes 230-250 on the acquired ultrasound image data 312, 322, 412, 422, 512, 522. The annotated settings changes 316, 326, 416, 426, 516, 526, annotated impact level 328, 428, 528, and the ultrasound examination information and/or at least some of the ultrasound scanning settings 314, 324, 414, 424, 514, 524 provides the ultrasound operator additional information regarding ultrasound scanning settings changes 230-250. The video clip 312, 322, 412, 422, 512, 522 may be presented at a display system 134 separately and/or with the log file 200 generated by the log file processor 150. The video clip 312, 322, 412, 422, 512, 522 may be stored at archive 138 and/or any suitable data storage medium.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present log files 200, video clips 312, 322, 412, 422, 512, 522 spanning a predetermined period of time before 310, 410, 510 and after 320, 420, 520 a settings change, ultrasound examination information 210, 314, 324, 414, 424, 514, 524, initial ultrasound scanning settings 220, ultrasound scanning settings changes 230-250, 316, 326, 416, 426, 516, 526, impact levels 230-250, 628, 428, 528, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores log files 200, video clips 312, 322, 412, 422, 512, 522 spanning a predetermined period of time before 310, 410, 510 and after 320, 420, 520 a settings change, instructions for detecting settings changes, instructions for generating log files 200, instructions for determining and assigning impact levels, instructions for generating video clips, and/or any suitable information, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

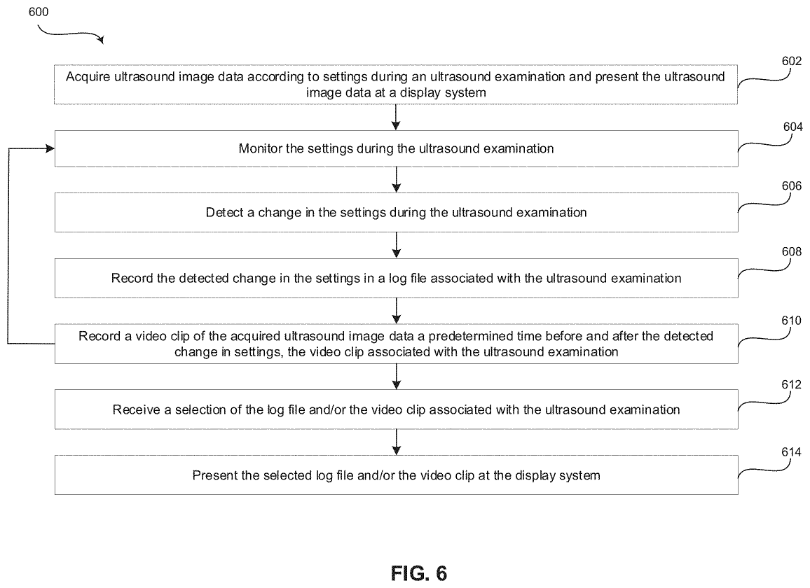

FIG. 6 is a flow chart 600 illustrating exemplary steps 602-614 for detecting changes to ultrasound scanning settings during an ultrasound examination and generating a record 200-500 of the settings changes, in accordance with various embodiments. Referring to FIG. 6, there is shown a flow chart 600 comprising exemplary steps 602 through 614. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 602, an ultrasound system 100 acquires ultrasound image data according to settings during an ultrasound examination and presents the ultrasound image data at a display system 134. For example, an ultrasound probe 104 of the ultrasound system 100 may be positioned to acquire ultrasound data in a region of interest. The ultrasound image data may be acquired by the ultrasound probe 104 according to ultrasound scanning settings. The ultrasound scanning settings initially applied by the ultrasound system 100 may be associated with a selected preset or otherwise provided by an ultrasound operator via the user input device 130. In various embodiments, the ultrasound operator may select an automatic optimization mode where ultrasound scanning settings are automatically changed by the ultrasound system 100 during the ultrasound examination. Additionally and/or alternatively, the ultrasound operator may manually change ultrasound scanning settings during the ultrasound examination. The ultrasound scanning settings may include, for example, a selected preset, a frequency setting, a gain setting, a depth setting, and any suitable initial settings selected by an ultrasound operator and/or included in a selected preset.

At step 604, at least one signal processor 132 of the ultrasound system 100 monitors the settings during the ultrasound examination. For example, a settings processor 140 of the at least one signal processor 132 may be configured to provide initial ultrasound scanning settings, such as settings associated with a selected preset or otherwise provided by an ultrasound operator via the user input device 130 to a log file processor 150 of the at least one signal processor 132. The settings processor 150 may be configured to monitor the ultrasound scanning settings to detect ultrasound scanning settings changes provided by the ultrasound system 100 when operating in an automatic optimization mode during the ultrasound examination. The settings processor 140 may be configured to monitor the ultrasound scanning settings to detect ultrasound scanning settings changes provided manually by an ultrasound operator via the user input device 130 during the ultrasound examination. In various embodiments, the settings processor 140 may selectively monitor ultrasound scanning settings based on whether the ultrasound scanning settings changes detection and recording feature is in an on state or an off state. For example, an ultrasound operator may turn on the ultrasound scanning settings changes detection and recording feature manually, such as to record short sequences of the ultrasound examination for teaching purposes. As another example, the ultrasound scanning settings changes detection and recording feature may be automatically placed in an on state based on pre-defined ultrasound operator selections, such as an ultrasound operator switching to a heart imaging preset.

At step 606, the at least one signal processor 132 of the ultrasound system 100 detects a change in the settings during the ultrasound examination. For example, the settings processor 140 of the at least one signal processor 132 may be configured to detect ultrasound scanning settings changes in response to monitoring the ultrasound scanning settings at step 604. The settings processor 140 may be configured to provide the detected settings changes to the log file processor 150 and a video clip processor 160 of the signal processor 132.

At step 608, the at least one signal processor 132 of the ultrasound system 100 records the detected change in the settings in a log file 200 associated with the ultrasound examination. For example, a log file processor 150 of the at least one signal processor 132 may be configured to receive the initial ultrasound scanning settings selected at the onset of an ultrasound examination from the settings processor 140. The log file processor 150 may be configured to generate a log file 200 and associate the log file 200 with the ultrasound examination. The log file processor 150 may be configured to record ultrasound examination information and the initial ultrasound scanning settings in the log file 200. The log file processor 150 may receive ultrasound scanning settings changes throughout the ultrasound examination from the settings processor 140 and may record each of the ultrasound scanning settings changes in the log file 200. The log file processor 150 may determine and assign an impact level of each settings change to an accuracy of a diagnosis based on the particular setting being changed and contextual information related to the ultrasound examination. The log file processor 150 may record the assigned impact level of each ultrasound scanning settings change in the log file 200. In various embodiments, the log file processor 150 may be configured to selectively record ultrasound scanning settings changes based on a pre-defined filter criteria. For example, the log file processor 150 may be configured to only record ultrasound scanning settings changes assigned a high impact level, or any suitable pre-defined filter criteria.

At step 610, the at least one signal processor 132 of the ultrasound system 100 records a video clip 312, 322, 412, 422, 512, 522 of the acquired ultrasound image data a predetermined time before and after the detected change in settings, the video clip 312, 322, 412, 422, 512, 522 associated with the ultrasound examination. For example, a video clip processor 160 of the at least one signal processor 132 may be configured to receive an indication of a settings change from the settings processor 140 at step 606 and in response, may record a video clip 312, 322, 412, 422, 512, 522 that begins 1-3 seconds (or any suitable defined period of time) before the settings change and ends 1-3 seconds (or any suitable defined period of time) after the settings change. The video clip processor 160 may be configured to stitch together each video clip corresponding with each ultrasound scanning settings change 230-250 to generate a continuous video clip illustrating all of the ultrasound scanning settings changes during an ultrasound examination. The video clip processor 160 may be configured to annotate the video clip(s) with the ultrasound scanning settings change, the assigned impact level, and/or any suitable information. The resulting video clip and/or the individual video clips may be associated with the ultrasound examination. In various embodiments, the video clip processor 160 may be configured to selectively record ultrasound scanning settings changes based on a pre-defined filter criteria. For example, the video clip processor 160 may be configured to only record ultrasound scanning settings changes in response to an indication from the log file processor 150 that the setting change was assigned a high impact level, or any suitable pre-defined filter criteria.

The process may repeat steps 604 through 610 until the ultrasound examination is completed.

At step 612, the at least one signal processor 132 of the ultrasound system 100 receives a selection of the log file 200 and/or the video clip 312, 322, 412, 422, 512, 522 associated with the ultrasound examination. For example, an ultrasound operator or application specialist may provide a selection of a log file 200 and/or video clip 312, 322, 412, 422, 512, 522 via the user input device 130 to review the ultrasound scanning settings changes made during a particular ultrasound examination.

At step 614, the at least one signal processor 132 of the ultrasound system 100 causes the display system 134 to present the selected log file 200 and/or the video clip 312, 322, 412, 422, 512, 522. For example, the selected log file 200 and/or video clip 312, 322, 412, 422, 512, 522 associated with the ultrasound examination may be retrieved by the at least one signal processor 132 and presented at the display system 134. The log file 200 and/or video clip 312, 322, 412, 422, 512, 522 may be reviewed by ultrasound operators to learn the relationship between particular ultrasound scanning settings changes and the effect on the present ultrasound image data. Ultrasound teachers may distribute the record of ultrasound scanning changes to students to review and memorize scanning activities. Application specialists may review the record of ultrasound scanning changes to identify and store customer image quality preferences and workflows. For example, the machine readable log file may be applied to the ultrasound system 100 to create new presets or modify existing presets.

Aspects of the present disclosure provide a method 600 and system 100 for detecting changes to ultrasound scanning settings during an ultrasound examination and generating a record 200, 300, 400, 500 of the settings changes. In accordance with various embodiments, the method 600 may comprise acquiring 602, by an ultrasound system 100, ultrasound image data according to ultrasound scanning settings during an ultrasound examination. The method 600 may comprise monitoring 604, by at least one processor 132, 140 of the ultrasound system 100, the ultrasound scanning settings during the ultrasound examination. The method 600 may comprise detecting 606, by the at least one processor 132, 140, a changed ultrasound scanning setting 230-250 from the ultrasound scanning setting during the ultrasound examination. The method 600 may comprise recording 608, by the at least one processor 132, 150, the changed ultrasound scanning setting 230-250 in a log file 200 associated with the ultrasound examination. The method 600 may comprise recording 610, by the at least one processor 132, 160, a video clip 312, 322, 412, 422, 512, 522 of the ultrasound image data a predetermined period of time before 310, 410, 510 and after 320, 420, 520 the changed ultrasound scanning setting 230-250 is detected. The video clip 312, 322, 412, 422, 512, 522 is associated with the ultrasound examination.

In a representative embodiment, the changed ultrasound scanning setting 230-250 is provided by the ultrasound system 100 operating in an automatic optimization mode. In an exemplary embodiment, the changed ultrasound scanning setting 230-250 is provided manually via a user input device 130. In various embodiments, the method 600 may comprise detecting 606, by the at least one processor 132, 140, an additional changed ultrasound scanning setting 230-250 from the ultrasound scanning setting during the ultrasound examination. The method 600 may comprise recording 608, by the at least one processor 132, 150, the additional changed ultrasound scanning setting 230-250 in the log file 200 associated with the ultrasound examination. The method 600 may comprise recording 610, by the at least one processor 132, 160, an additional video clip 312, 322, 412, 422, 512, 522 of the ultrasound image data the predetermined period of time before 310, 410, 510 and after 320, 420, 520 the additional changed ultrasound scanning setting 230-250 is detected. The additional video clip 312, 322, 412, 422, 512, 522 is associated with the ultrasound examination. The method 600 may comprise stitching together 610, by the at least one processor 132, 160, the video clip 312, 322, 412, 422, 512, 522 and the additional video clip 312, 322, 412, 422, 512, 522 to generate a single video clip 312, 322, 412, 422, 512, 522 associated with the ultrasound examination. In certain embodiments, the method 600 may comprise receiving 612, by the at least one processor 132, a selection of one or both of the log file 200 or the single video clip. The method 600 may comprise presenting 614 the one or both of the log file 200 or the single video clip 312, 322, 412, 422, 512, 522 at a display system 134 in response to the selection. In a representative embodiment, the method 600 may comprise assigning 608, by the at least one processor 132, 150, an impact level 328, 428, 528 of the changed ultrasound scanning setting 230-250 to an accuracy of a diagnosis based on an identity of the changed ultrasound scanning setting 230-250 and ultrasound examination contextual information. The method 600 may comprise recording 608, by the at least one processor 132, 150, the impact level 328, 428, 528 in the log file 200 with the changed ultrasound scanning setting 230-250. In an exemplary embodiment, the method 600 may comprise annotating 610, by the at least one processor 132, 160, the video clip 312, 322, 412, 422, 512, 522 to include the changed ultrasound scanning setting 316, 326, 416, 426, 516, 526 and the impact level 328, 428, 528. In certain embodiments, the method 600 comprises applying 614 the log file 200 to the ultrasound system 100 to one or both of create a new preset, or modify an existing preset. The log file 200 is machine readable.

Various embodiments provide a system 100 for detecting changes to ultrasound scanning settings during an ultrasound examination and generating a record 200, 312, 322, 412, 422, 512, 522 of the settings changes. The system may comprise an ultrasound system 100 and at least one processor 132, 140, 150, 160. The ultrasound system 100 may be configured to acquire ultrasound image data according to ultrasound scanning settings during an ultrasound examination. The at least one processor 132, 140 may be configured to monitor the ultrasound scanning settings during the ultrasound examination. The at least one processor 132, 140 may be configured to detect a changed ultrasound scanning setting 230-250 from the ultrasound scanning setting during the ultrasound examination. The at least one processor 132, 150 may be configured to record the changed ultrasound scanning setting 230-250 in a log file 200 associated with the ultrasound examination. The at least one processor 132, 160 may be configured to record a video clip 312, 322, 412, 422, 512, 522 of the ultrasound image data a predetermined period of time before and after the changed ultrasound scanning setting 230-250 is detected. The video clip 312, 322, 412, 422, 512, 522 is associated with the ultrasound examination.

In an exemplary embodiment, the changed ultrasound scanning setting 230-250 is provided by one of manually via a user input device 130, or automatically by the ultrasound system 100 operating in an automatic optimization mode. In various embodiments, the at least one processor 132, 140 is configured to detect an additional changed ultrasound scanning setting 230-250 from the ultrasound scanning setting during the ultrasound examination. The at least one processor 132, 150 is configured to record the additional changed ultrasound scanning setting 230-250 in the log file 200 associated with the ultrasound examination. The at least one processor 132, 160 is configured to record an additional video clip 312, 322, 412, 422, 512, 522 of the ultrasound image data the predetermined period of time before and after the additional changed ultrasound scanning setting 230-250 is detected. The additional video clip 312, 322, 412, 422, 512, 522 is associated with the ultrasound examination. The at least one processor 132, 160 is configured to stitch together the video clip 312, 322, 412, 422, 512, 522 and the additional video clip 312, 322, 412, 422, 512, 522 to generate a single video clip 312, 322, 412, 422, 512, 522 associated with the ultrasound examination. In certain embodiment, the system comprises a display system 134. The at least one processor 132 is configured to receive a selection of one or both of the log file 200 or the single video clip 312, 322, 412, 422, 512, 522. The at least one processor 132 is configured to cause the display system 134 to present the one or both of the log file 200 or the single video clip 312, 322, 412, 422, 512, 522 in response to the selection. In a representative embodiment, the at least one processor 132, 150 is configured to assign an impact level 328, 428, 528 of the changed ultrasound scanning setting 230-250 to an accuracy of a diagnosis based on an identity of the changed ultrasound scanning setting 230-250 and ultrasound examination contextual information. The at least one processor 132, 150 is configured to record the impact level 328, 428, 528 in the log file 200 with the changed ultrasound scanning setting 230-250. In an exemplary embodiment, the at least one processor 132, 160 is configured to annotate the video clip 312, 322, 412, 422, 512, 522 to include the changed ultrasound scanning setting 230-250, 316, 326, 416, 426, 516, 526 and the impact level 328, 428, 528. In various embodiments, the log file 200 is machine readable. The at least one processor 132 is configured to apply the log file 200 to the ultrasound system 100 to one or both of create a new preset, or modify an existing preset.

Certain embodiments provide a non-transitory computer readable medium having stored thereon, a computer program having at least one code section. The at least one code section is executable by a machine for causing an ultrasound system 100 to perform steps 600. The steps 600 may comprise receiving 602 ultrasound image data acquired according to ultrasound scanning settings during an ultrasound examination. The steps 600 may comprise monitoring 604 the ultrasound scanning settings during the ultrasound examination. The steps 600 may comprise detecting 606 a changed ultrasound scanning setting 230-250 from the ultrasound scanning setting during the ultrasound examination. The steps 600 may comprise recording 608 the changed ultrasound scanning setting 230-250 in a log file 200 associated with the ultrasound examination. The steps 600 may comprise recording 610 a video clip 312, 322, 412, 422, 512, 522 of the ultrasound image data a predetermined period of time before 310, 410, 510 and after 320, 420, 520 the changed ultrasound scanning setting 230-250 is detected. The video clip 312, 322, 412, 422, 512, 522 is associated with the ultrasound examination.

In various embodiments, the changed ultrasound scanning setting 230-250 is provided by one of the ultrasound system 100 operating in an automatic optimization mode, or manually via a user input device 130. In certain embodiments, the steps 600 may comprise detecting 606 an additional changed ultrasound scanning setting 230-250 from the ultrasound scanning setting during the ultrasound examination. The steps 600 may comprise recording 608 the additional changed ultrasound scanning setting 230-250 in the log file 200 associated with the ultrasound examination. The steps 600 may comprise recording 610 an additional video clip 312, 322, 412, 422, 512, 522 of the ultrasound image data the predetermined period of time before 310, 410, 510 and after 320, 420, 520 the additional changed ultrasound scanning setting 230-250 is detected. The additional video clip 312, 322, 412, 422, 512, 522 is associated with the ultrasound examination. The steps 600 may comprise stitching together 610 the video clip 312, 322, 412, 422, 512, 522 and the additional video clip 312, 322, 412, 422, 512, 522 to generate a single video clip 312, 322, 412, 422, 512, 522 associated with the ultrasound examination. In a representative embodiment, the steps 600 may comprise assigning 608 an impact level 328, 428, 528 of the changed ultrasound scanning setting 230-250 to an accuracy of a diagnosis based on an identity of the changed ultrasound scanning setting 230-250 and ultrasound examination contextual information. The steps 600 may comprise recording 608 the impact level 328, 428, 528 in the log file 200 with the changed ultrasound scanning setting 230-250. In an exemplary embodiment, the steps 600 may comprise annotating 610 the video clip 312, 322, 412, 422, 512, 522 to include the changed ultrasound scanning setting 230-250 316, 326, 416, 426, 516, 526 and the impact level 328, 428, 528.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for detecting changes to ultrasound scanning settings during an ultrasound examination and generating a record of the settings changes.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
acquiring, by an ultrasound system, ultrasound image data according to ultrasound scanning settings during an ultrasound examination;
monitoring, by at least one processor of the ultrasound system, the ultrasound scanning settings during the ultrasound examination;
detecting, by the at least one processor, a changed ultrasound scanning setting from the ultrasound scanning setting during the ultrasound examination;
recording, by the at least one processor, the changed ultrasound scanning setting in a log file associated with the ultrasound examination; and
recording, by the at least one processor, a video clip of the ultrasound image data a predetermined period of time before and after the changed ultrasound scanning setting is detected, the video clip associated with the ultrasound examination.

2. The method of claim 1, wherein the changed ultrasound scanning setting is provided by the ultrasound system operating in an automatic optimization mode.

3. The method of claim 1, wherein the changed ultrasound scanning setting is provided manually via a user input device.

4. The method of claim 1, comprising:
detecting, by the at least one processor, an additional changed ultrasound scanning setting from the ultrasound scanning setting during the ultrasound examination;
recording, by the at least one processor, the additional changed ultrasound scanning setting in the log file associated with the ultrasound examination;
recording, by the at least one processor, an additional video clip of the ultrasound image data the predetermined period of time before and after the additional changed ultrasound scanning setting is detected, the additional video clip associated with the ultrasound examination; and
stitching together, by the at least one processor, the video clip and the additional video clip to generate a single video clip associated with the ultrasound examination.

5. The method of claim 4, comprising:
receiving, by the at least one processor, a selection of one or both of the log file or the single video clip; and
presenting the one or both of the log file or the single video clip at a display system in response to the selection.

6. The method of claim 1, comprising:
assigning, by the at least one processor, an impact level of the changed ultrasound scanning setting to an accuracy of a diagnosis based on an identity of the changed ultrasound scanning setting and ultrasound examination contextual information; and
recording, by the at least one processor, the impact level in the log file with the changed ultrasound scanning setting.

7. The method of claim 6, comprising annotating, by the at least one processor, the video clip to include the changed ultrasound scanning setting and the impact level.

8. The method of claim 1, comprising applying the log file to the ultrasound system to one or both of create a new preset, or modify an existing preset, wherein the log file is machine readable.

9. A system comprising:
an ultrasound system configured to acquire ultrasound image data according to ultrasound scanning settings during an ultrasound examination; and
at least one processor configured to:
monitor the ultrasound scanning settings during the ultrasound examination;
detect a changed ultrasound scanning setting from the ultrasound scanning setting during the ultrasound examination;
record the changed ultrasound scanning setting in a log file associated with the ultrasound examination; and
record a video clip of the ultrasound image data a predetermined period of time before and after the changed ultrasound scanning setting is detected, the video clip associated with the ultrasound examination.

10. The system of claim 9, wherein the changed ultrasound scanning setting is provided by one of manually via a user input device, or automatically by the ultrasound system operating in an automatic optimization mode.

11. The system of claim 9, wherein the at least one processor is configured to:
detect an additional changed ultrasound scanning setting from the ultrasound scanning setting during the ultrasound examination;
record the additional changed ultrasound scanning setting in the log file associated with the ultrasound examination;
record an additional video clip of the ultrasound image data the predetermined period of time before and after the additional changed ultrasound scanning setting is detected, the additional video clip associated with the ultrasound examination; and
stitch together the video clip and the additional video clip to generate a single video clip associated with the ultrasound examination.

12. The system of claim 11, comprising a display system, wherein the at least one processor is configured to:
receive a selection of one or both of the log file or the single video clip; and
cause the display system to present the one or both of the log file or the single video clip in response to the selection.

13. The system of claim 9, wherein the at least one processor is configured to:
assign an impact level of the changed ultrasound scanning setting to an accuracy of a diagnosis based on an identity of the changed ultrasound scanning setting and ultrasound examination contextual information; and
record the impact level in the log file with the changed ultrasound scanning setting.

14. The system of claim 13, wherein the at least one processor is configured to annotate the video clip to include the changed ultrasound scanning setting and the impact level.

15. The system of claim 9, wherein:
the log file is machine readable; and
the at least one processor is configured to apply the log file to the ultrasound system to one or both of create a new preset, or modify an existing preset.

16. A non-transitory computer readable medium having stored thereon, a computer program having at least one code section, the at least one code section being executable by a machine for causing an ultrasound system to perform steps comprising:
receiving ultrasound image data acquired according to ultrasound scanning settings during an ultrasound examination;
monitoring the ultrasound scanning settings during the ultrasound examination;
detecting a changed ultrasound scanning setting from the ultrasound scanning setting during the ultrasound examination;
recording the changed ultrasound scanning setting in a log file associated with the ultrasound examination; and
recording a video clip of the ultrasound image data a predetermined period of time before and after the changed ultrasound scanning setting is detected, the video clip associated with the ultrasound examination.

17. The non-transitory computer readable medium of claim 16, wherein the changed ultrasound scanning setting is provided by one of:
the ultrasound system operating in an automatic optimization mode, or
manually via a user input device.

18. The non-transitory computer readable medium of claim 16, comprising:
detecting an additional changed ultrasound scanning setting from the ultrasound scanning setting during the ultrasound examination;
recording the additional changed ultrasound scanning setting in the log file associated with the ultrasound examination;
recording an additional video clip of the ultrasound image data the predetermined period of time before and after the additional changed ultrasound scanning setting is detected, the additional video clip associated with the ultrasound examination; and
stitching together the video clip and the additional video clip to generate a single video clip associated with the ultrasound examination.

19. The non-transitory computer readable medium of claim 16, comprising:
assigning an impact level of the changed ultrasound scanning setting to an accuracy of a diagnosis based on an identity of the changed ultrasound scanning setting and ultrasound examination contextual information; and
recording the impact level in the log file with the changed ultrasound scanning setting.

20. The non-transitory computer readable medium of claim 19, comprising annotating the video clip to include the changed ultrasound scanning setting and the impact level.
